(19) United States
(12) Patent Application Publication
(10) Pub. No.: US 2007/0299467 A1
(43) Pub. Date: Dec. 27, 2007

(54) Tourniquet

(76) Inventor: Jose Arias, Miami, FL (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/751,956

(22) Filed: May 22, 2007

Related U.S. Application Data
(60) Provisional application No. 60/805,391, filed on Jun. 21, 2006.

(51) Int. Cl. A61B 17/00

(52) U.S. Cl. 606/203

(57) ABSTRACT

A tourniquet may include an elastic band having a first end and a second end, a first attachment member affixed at the first end, a second attachment member affixed at the second end; and a third attachment member, which may be attached to and slideably displaceable along the elastic band. The third attachment member may be sized and shaped to be attached to the first attachment member and the second attachment member. The third attachment member may be held to the band by a resistive element that inhibits displacement of the third attachment element caused by gravity.

### CROSS-REFERENCE TO RELATED APPLICATIONS

This application claims the benefit of U.S. Provisional Application No. 60/805,391, filed Jun. 21, 2006, which is hereby incorporated herein by reference.

### SUMMARY

The present disclosure describes tourniquets and methods of creating hemostasis in a limb using the tourniquets.

In an embodiment, a tourniquet may include an elastic band having a first end and a second end, a first attachment member affixed at the first end, a second attachment member affixed at the second end; and a third attachment member, which may be attached to and slideably displaceable along the elastic band. The third attachment member may be sized and shaped to be attached to the first attachment member and the second attachment member. The third attachment member may be held to the band by a resistive element that inhibits displacement of the third attachment element caused by gravity.

In an embodiment, a method of creating hemostasis in a limb may include draping a tourniquet disclosed herein over the limb, attaching the first attachment member to the third attachment member, stretching the elastic band so that the portion draped over the limb snugly contacts the limb, wrapping the elastic band around the limb with sufficient force so that the pressure exerted by the band upon the limb creates hemostasis in the limb, continuing wrapping until sufficiently little slack remains in the limb so that, after the second attachment member is attached to the third attachment member, the elastic band cannot so slacken as to disturb the hemostasis, and attaching the second attachment member to the third attachment member.

### BRIEF DESCRIPTION OF THE DRAWINGS

FIG. 1 schematically depicts one exemplary embodiment of a tourniquet.

FIG. 2 is a photograph of an embodiment of a tourniquet.

FIGS. 3-9 are photographs depicting steps in an exemplary method of creating hemostasis in a limb.

### DETAILED DESCRIPTION

Tourniquets are applied to limbs to stop bleeding. They are typically applied to a limb proximal to an exsanguination site and sufficiently tightened to overcome the blood pressure driving the bleeding (typically arterial), thereby stopping the bleeding. Tourniquets are also used to assist in phlebotomy; for this use, the tourniquet is tightened just enough to permit blood to collect in the vein to be accessed.

Tourniquets disclosed herein may be used for these purposes. In some embodiments, they may be applied one-handed. Consequently, they may be especially well-suit for use in emergency situations, such as when a person who is in need of tourniquet application is isolated or has no immediate access to medical personnel or to another person to apply the tourniquet.

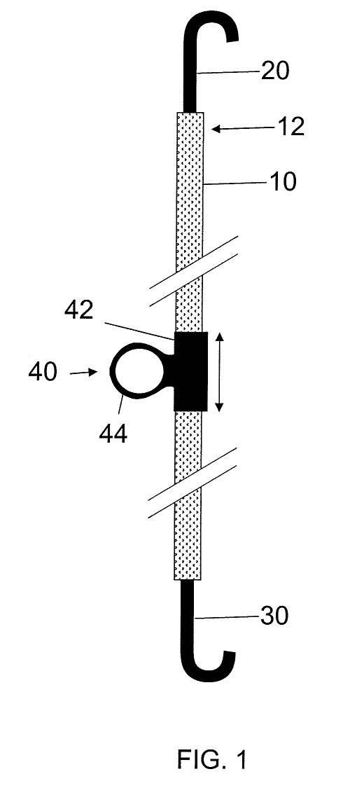

FIG. 1 depicts schematically an exemplary tourniquet. The tourniquet includes an elastic band 10, a first attachment member 20 at a first end 12 of the band, a second attachment member 30 and a second end 14 of the band, and a third attachment member 40 that is slideably displaceable along the band. The third attachment member may include a portion 42 that holds it to the band and makes it slideably displaceable along the band, and a portion 44 that can receive the first attachment member and/or second attachment member. Although the first and second attachment members are depicted in

FIG. 1 as hooks and the third attachment member is depicted as a ring, there are merely exemplary shapes for illustrative purposes. The attachment members may have a wide variety of shapes and may take the form of, for example, hooks, eyelets, clasps, etc.

One of the chief difficulties in applying a tourniquet one-handed is getting the tourniquet in snug contact with the limb. The tourniquet disclosed herein may overcome this difficulty by providing a member that can slide along the tourniquet. When the tourniquet is draped over/around a limb and the sliding member is attached to one end, the other end of the tourniquet may be tugged; this takes up the slack around the limb and makes the tourniquet snug around the limb.

The elastic band may be made from a wide variety of materials, such as latex rubber and other elastic polymers. The band may include a bungee cord. The band may be provided in a variety of shapes, such as a sheet, a cord, and/or a strap. The band may have rounded edges to help prevent discomfort or tissue injury. The band should be long enough to allow it to be wrapped around a limb, such as a human upper extremity, arm, forearm, lower extremity, thigh, or leg. The band should be stretchable so that it may be wrapped around the limb and thereby compress the limb. The band should have a spring constant such that it may be stretched enough to exert sufficient pressure on a limb to exceed the blood pressure causing bleeding. The spring constant may be in the range of, for example, about 1 N/m to about 300 N/m. A tourniquet used to control arterial bleeding should be able to exert enough pressure to overcome the arterial systolic pressure; accordingly, the stretched elastic band should be able to overcome 80 mm Hg, 100 mmHg, 120 mmHg, 150 mmHg, and/or 200 mmHg. The band may have a width of at least ¼ inch, at least ½ inch, at least 1 inch, up to 1½ inches, up to 2 inches, between 1 inch and 2 inches, and/or between 1 inch and 1½ inches.

The attachment members can be made from a variety of materials, such as metals, plastics, and/or polymers.

The third attachment member may be shaped to resist free motion along the band. For example, portion 42 may closely grip the band so that the third attachment member moves only when tugged with a minimum force, such as 5 N, 10N, 20N, 50N, and/or 100 N. In one embodiment, the portion 42 and/or may include an adhesive surface. In another embodiment, a surface of the portion 42 may include hook- or loop-fasteners, and the band may include the complementary material.

### EXAMPLE

The present description is further illustrated by the following example, which should not be construed as limiting the claims in any way.

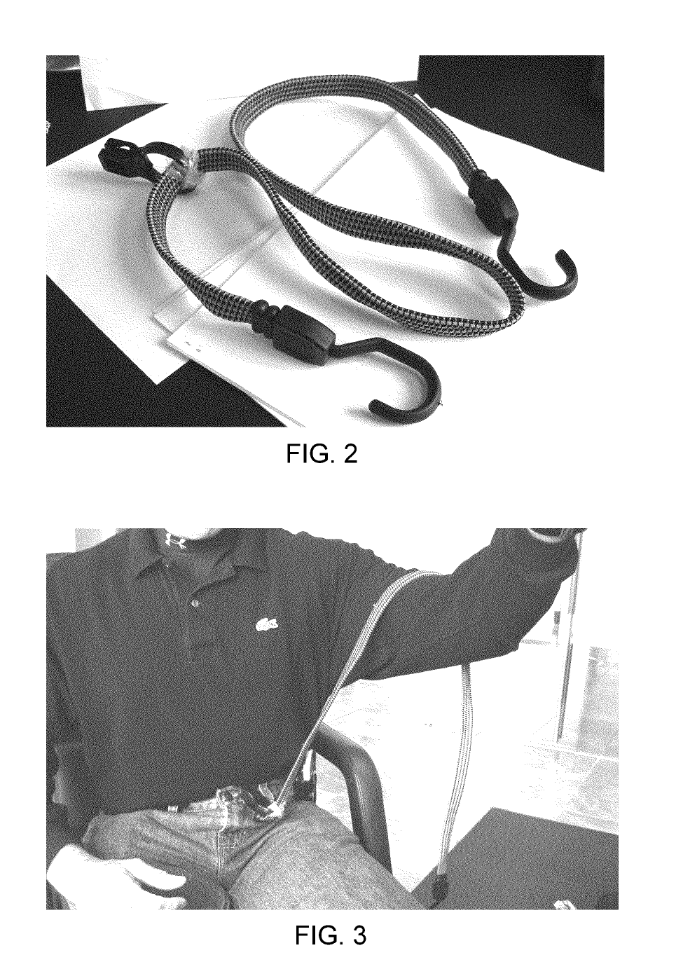

FIG. 2 is photograph of an exemplary embodiment of a tourniquet. It includes a bungee cord (HIGHLAND brand “Fat Strap” bungee cord, about ¾″ wide, ⅛″ thick, and about three feet long) with plastic-covered metal hooks on either end. A sliding ring is affixed to the cord with a loop of adhesive tape.

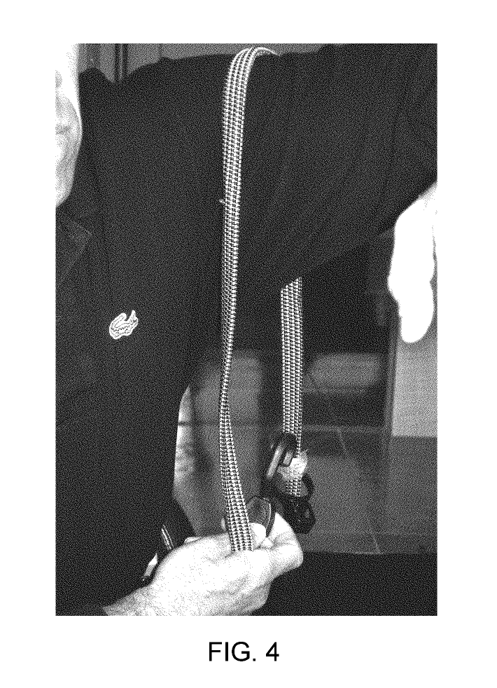

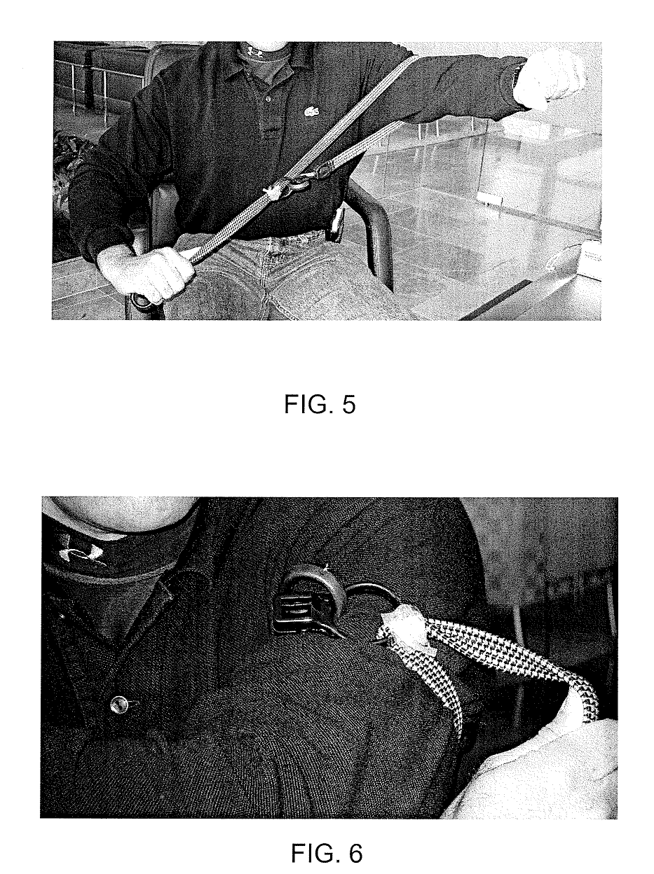

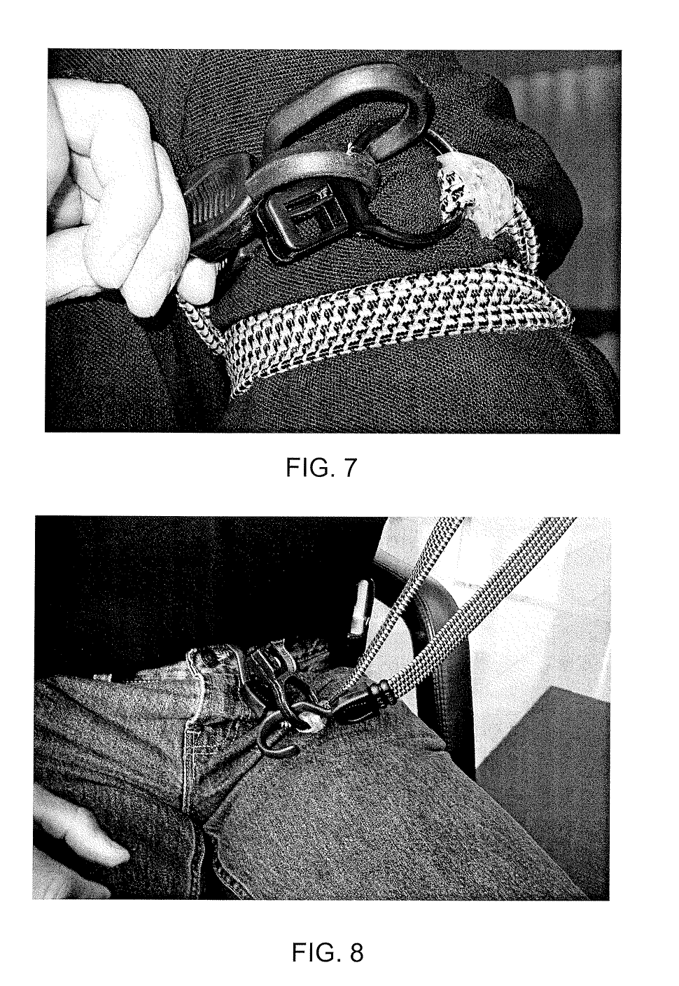

FIGS. 3-9 depict steps in an exemplary method of creating limb hemostasis. The tourniquet is draped over the limb (FIG. 3). One end is hooked to the slider (FIG. 4). The other end is pulled (FIG. 5) until the tourniquet is snug around the limb (FIG. 6). The tourniquet is then tightly wrapped around the limb to compress it, and the other end is hooker to the slider (FIG. 7). The entire process may be carried out with one hand.

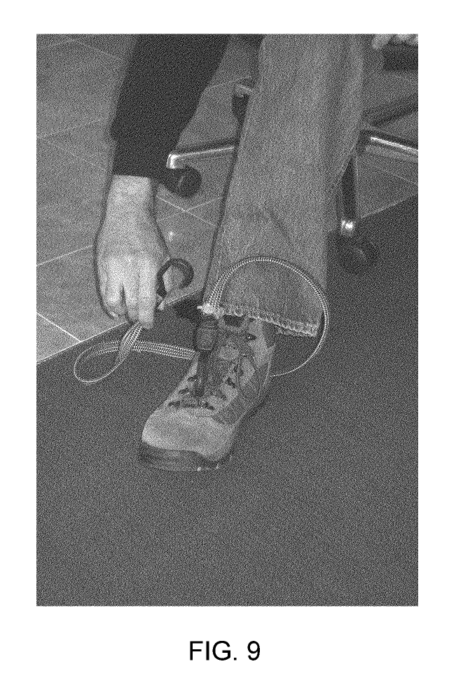

In some instances, one end of the tourniquet may be anchored to facilitate draping the tourniquet and attaching one end to the slider. Suitable anchors include, for example, a belt loop when applying the tourniquet to an arm (FIG. 8) or a shoelace when applying the tourniquet to a leg (FIG. 9).

I claim:

1. A tourniquet comprising:
an elastic band having a first end and a second end;
a first attachment member affixed at the first end;
a second attachment member affixed at the second end; and
a third attachment member, which is:
attached to and slideably displaceable along the elastic band; and
sized and shaped to be attached to the first attachment member and the second attachment member.

2. The tourniquet of claim 1, wherein the elastic band has a spring constant so high as to exert at least 100 mmHg pressure upon a limb when the band is wrapped around the limb.

3. The tourniquet of claim 1, wherein the elastic band has a spring constant so high as to exert at least 150 mmHg pressure upon a limb when the band is wrapped around the limb.

4. The tourniquet of claim 1, wherein the elastic band has a spring constant so high as to exert at least 200 mmHg pressure upon a limb when the band is wrapped around the limb.

5. The tourniquet of claim 1, wherein the first attachment member and the second attachment member each comprise a hook.

6. The tourniquet of claim 1, wherein the third attachment member comprises a first loop configured to receive the first and second attachment members.

7. The tourniquet of claim 6, wherein the third attachment member further comprises a second loop configured to allow the third attachment member to slide along the elastic band.

8. The tourniquet of claim 7, wherein the second loop attaches so snugly around the elastic band as to inhibit displacement of the third attachment member due to gravity.

9. The tourniquet of claim 1, wherein the band is formed at least in part by latex.

10. The tourniquet of claim 1, wherein the band comprises latex strands.

11. The tourniquet of claim 1, wherein the band comprises a latex sheet.

12. The tourniquet of claim 1, wherein the third attachment member comprises a ratchet.

13. A tourniquet comprising:
an elastic band having a first end and a second end;
a first attachment member affixed at the first end;
a second attachment member affixed at the second end; and
a third attachment member slideably displaceable along the elastic band and held to the band by a resistive element that inhibits displacement of the third attachment element caused by gravity.

14. A method of creating hemostasis in a limb comprising:
draping over the limb a tourniquet as defined by claim 1;
attaching the first attachment member to the third attachment member;
stretching the elastic band so that the portion draped over the limb snugly contacts the limb;
wrapping the elastic band around the limb with sufficient force so that the pressure exerted by the band upon the limb creates hemostasis in the limb;
continuing wrapping until sufficiently little slack remains in the limb so that, after the second attachment member is attached to the third attachment member, the elastic band cannot so slacken as to disturb the hemostasis; and
attaching the second attachment member to the third attachment member.

15. The method of claim 14, wherein draping comprises draping the first end of the elastic band over a first side of the limb, and wrapping comprises wrapping the second end of the elastic band over the same side of the limb.

16. The method of claim 14, wherein the limb comprises an arm.

17. The method of claim 14, wherein the limb comprises a thigh.

18. The method of claim 14, further comprising attaching the second attachment member to an anchor point before draping, and removing the second attachment member from the anchor point after attaching the first attachment member to the third attachment member.

19. The method of claim 14, wherein the steps are performed in the recited order.

20. The method of claim 14, wherein the steps are performed using only one hand.
